(19) United States
(12) Patent Application Publication
(10) Pub. No.: US 2011/0190763 A1
(43) Pub. Date: Aug. 4, 2011

(54) Needle Design for Recording Monophasic Action Potential and Delivery of Therapy

(75) Inventors: Jon F. Urban, Minneapolis, MN (US); Vinod Sharma, Blaine, MN (US); Mark T. Stewart, Lino Lakes, MN (US)

(73) Assignee: Medtronic, Inc., Minneapolis, MN (US)

(21) Appl. No.: 12/696,239

(22) Filed: Jan. 29, 2010

(51) Int. Cl. A61B 18/14

(52) U.S. Cl. 606/41

(57) ABSTRACT

A system and associated method measure monophasic action potential signals for identifying a targeted tissue location and delivering a therapy to the targeted tissue location. The system includes a hollow needle having a sharpened distal tip, a first electrode at the distal tip and a fluid delivery lumen extending through the needle from a proximal needle end to an opening in the sharpened distal tip.

### TECHNICAL FIELD

The disclosure relates generally to implantable medical devices and, in particular, to a method and apparatus for measuring monophasic action potential signals and delivering a fluid as a part of a therapy delivery process.

### BACKGROUND

Gene therapies, and other types of therapies, can be delivered to the heart to modify myocardial cells to treat cardiac arrhythmias, heart failure or other abnormalities. Such therapies are typically delivered using a catheter that is placed next to a cardiac electrogram (EGM) mapping catheter. EGM signals are first obtained using the mapping catheter to map an area of the myocardium and to approximate a targeted location to deliver a desired therapy. EGM signals, however, including bipolar EGM signals, have limited mapping resolution since EGM signals will often be sensed from a volume of myocardial tissue that is significantly larger than a cardiac structure or area of diseased tissue being targeted for the therapy delivery. Furthermore, EGM signals do not fully reveal all the electrical details of an action potential signal (e.g. morphology), which is the true signature of the electrical activity of a specific cardiac structure. As a result, the therapy delivered may not precisely reach a targeted area of cardiac cells, limiting the efficacy of the therapy, or a larger volume of tissue may need to be treated to achieve a desired result. A need remains, therefore, for apparatus and methods for precise identification of targeted therapy delivery sites and highly localized therapy delivery to an identified site.

### BRIEF DESCRIPTION OF THE DRAWINGS

FIG. 1 is a plan view of a cardiac catheter system for monitoring monophasic action potential (MAP) signals using a transvenous endocardial approach.

FIG. 2 is an enlarged view of a distal end of a hollow needle for measuring MAP signals according to an alternative embodiment.

FIG. 3 is an illustration of an EGM signal and a MAP signal.

FIG. 4 is an enlarged view of a distal end of a catheter including MAP measurement electrodes according to an alternative embodiment.

FIG. 5 is a sectional view of the catheter shown in FIG. 4.

FIG. 6 is a sectional view of a distal end of a catheter according to an alternative embodiment.

FIG. 7 is an enlarged plan view of a distal end of a catheter including an elongated catheter body.

FIG. 8 is a schematic view of a catheter system for measuring MAP signals and delivering an ablation therapy.

FIG. 9 is a flow chart of a method for delivering a therapy.

### DETAILED DESCRIPTION

In the following description, references are made to illustrative embodiments. It is understood that other embodiments may be utilized without departing from the scope of the disclosure. For purposes of clarity, identical reference numbers are used in the drawings to identify similar elements. Unless indicated, drawing elements are not shown to scale.

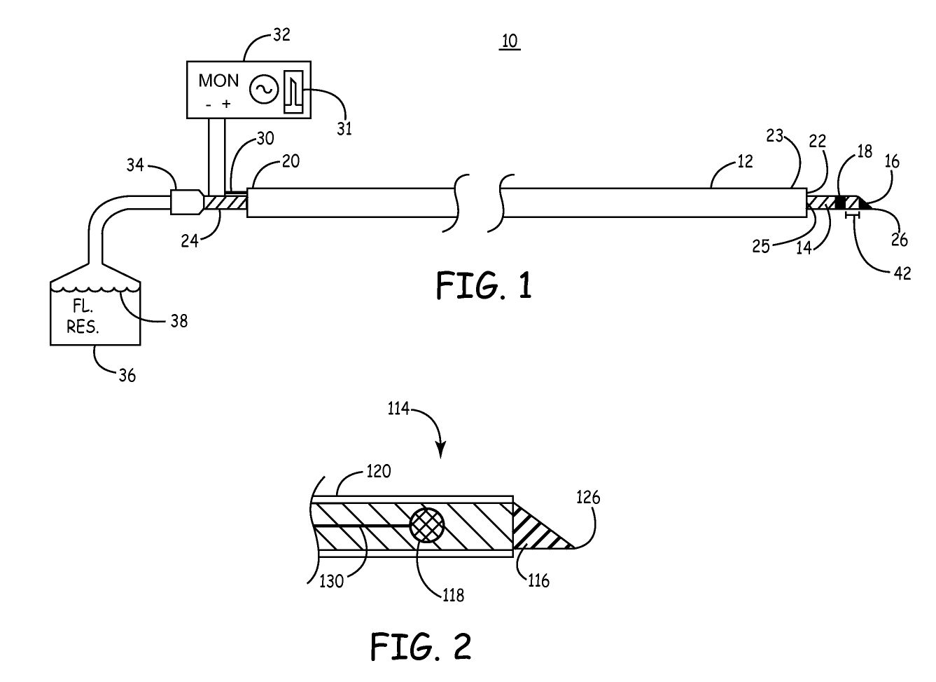

FIG. 1 is a plan view of a cardiac catheter system 10 for monitoring monophasic action potential (MAP) signals using a transvenous endocardial approach. It is recognized that various embodiments and methods described herein may alternatively be implemented in an epicardial approach to the heart. MAP signals are localized measurements of cellular depolarization that closely resemble the morphology of a single cell action potential. As compared to EGM signals, the relatively more localized MAP signals can be used to more precisely differentiate relatively small cardiac structures such as the sinoatrial (SA) node and the atrioventricular (AV) node, which both exhibit distinctly different signals than surrounding myocardial tissue. MAP signals can also be used to measure localized differences in myocardial tissue associated with pathological or biological processes or altered refractoriness. MAP signals can thus provide more precise localization of cardiac structures or boundaries of altered myocardial tissue for identification of a targeted therapy delivery site than EGM or ECG signals.

MAP signals are obtained by depolarizing tissue with one electrode, referred to herein as the “reference electrode”, while measuring the resulting electrical signal using a second electrode, referred to herein as the “recording electrode”. The recording electrode is positioned in close proximity to the reference electrode to obtain a highly localized MAP signal. Depolarization of the tissue at the reference electrode can be accomplished by injecting potassium chloride or by applying pressure. However, in clinical practice, application of pressure against the myocardial surface from a transvenous endocardial approach is challenging for a number of reasons. Application of pressure typically requires application of torque to an external end of a catheter and/or additional mechanisms which add design complexity to the catheter. The resulting pressure applied may not be stable during the measurement period. Injection of potassium chloride is generally undesirable in clinical applications because of the potential for arrhythmogenesis.

System 10 includes an elongated catheter 12 extending between a proximal catheter end 20 and a distal catheter end 23 terminated by a lateral distal face 22 of the catheter. Catheter 12 includes an open lumen (not shown in the plan view of FIG. 1) extending from the proximal end 20 to an opening 25 in the lateral distal face 22 of the catheter.

A hollow needle 14 extends through the open lumen of the catheter 12 and is advanceable out from opening 25. Hollow needle 14 includes a sharpened distal tip 26 for piercing tissue. A reference electrode 16 is formed at the distal tip 26. Hollow needle 14 may be formed of a conductive material, such as stainless steel. The outer surface of the conductive needle may be insulated with an electrically insulative coating, such as a polyurethane or polyethylene coating. An opening in the insulative coating at distal tip 26 may be provided to form reference electrode 16.

A recording electrode 18 is positioned proximally from reference electrode 16 along needle 14. Recording electrode 18 may be a ring electrode fixedly coupled to the outer surface of an insulative coating of needle 14 such that recording electrode 18 and reference electrode 16 are electrically insulated from each other. Reference electrode 16 may extend circumferentially around an entire circumference of needle 14 in the form of a ring electrode or, alternatively, extend non-circumferentially along any portion of the needle outer longitudinal surface, for example as a button or point electrode.

Both reference electrode 16 and recording electrode 18 may be formed as low polarization electrodes. Low polarization electrodes are formed of a material or coated with a material that reduces polarization artifact. Reference is made to U.S. Pat. No. 6,253,110 (Brabec, et al.), for illustrative examples of low polarization electrodes.

Needle tip 26 causes transient tissue injury to a highly localized region of the myocardium when the sharpened needle tip 26 is inserted into myocardial tissue. The tissue injury depolarizes the myocardial cells at reference electrode 16 formed at needle tip 26, enabling differential voltage MAP signals to be measured between the recording electrode 18 and the reference electrode 16.

The needle tip 26 is sized small enough to limit the area of injury. The injury may be a reversible injury that is sustained for a period of time long enough to allow desired measurements to be taken. Alternatively, the injury may be irreversible but limited in size so as to be clinically insignificant. In one embodiment, the needle tip 26 has an outer diameter of up to approximately 1 mm. In other embodiments, the needle tip 26 has an outer diameter of less than approximately 0.5 mm. For example, the approximate dimensions of needle 14 may correspond to a 27 gauge needle having an outer diameter of approximately 0.36 mm and an inner diameter, defining the opening of the hollow needle lumen, of approximately 0.23 mm.

The tissue injury that results in local depolarization of cells and subsequent inhibition of repolarization enables measurement of a highly localized MAP signal to be measured in contrast to standard EGM sensing electrodes which are not causing cellular depolarization nor inhibiting repolarization during EGM sensing. EGM sensing electrodes sense a relatively more global activation of the cardiac cells that occurs through the natural conduction system of the heart. The EGM signal represents a spatial averaging of the action potential signals over a measurement volume and losing the detailed electrical signal characteristics of MAP signals, which closely resemble an action potential signal. The global activation sensed by EGM electrodes may be initiated intrinsically, e.g. by the SA node, or by delivering a pacing pulse that is conducted naturally through the myocardial cells. Standard cardiac electrodes, including bipolar EGM sensing electrodes, are typically sized and spaced to obtain a relatively more global cardiac electrogram signal that is distinctly different than a MAP signal. Signals collected by standard bipolar EGM electrode configurations provide an area averaged signal that may emanate from a relatively large surface area of the endocardium. When targeting specific arrhythmogenic sites, this spatial resolution is insufficient to precisely locate a targeted site. Furthermore, the signal from bipolar electrograms only provides timing information and lacks the information on the duration of the local action potential in the underlying myocardium.

The sizing and spacing of reference electrode 16 and recording electrode 18 are selected to allow recording of MAP signals having an action-potential like morphology, thus providing the detail of the electrical signal morphology needed to precisely identify the type of tissue at the measurement site. Generally, the smaller the size and the closer the spacing of the electrodes the more localized the MAP signal. The spatial averaging of individual cellular signals becomes smaller as the spacing and size of the electrodes becomes smaller. However, the smaller and closer the electrodes becomes, the lower the signal strength will become. As such, there will generally be an optimal electrode spacing for given electrode surface areas that results in good signal to noise ratio and still retains the action potential-like signal morphology.

Commercially available cardiac leads typically provide bipolar EGM sensing electrodes spaced at least 2 mm apart and often 8 to 10 mm apart or more. The combination of the relatively larger spacing and larger electrode surface area results in an EGM signal that is the spatially-averaged over a relatively larger volume of myocardial cells than a MAP signal.

In order to obtain a MAP signal that is morphologically similar to a cellular action potential signal, the electrode spacing and electrode surface area is kept small and the reference electrode is used to induce a localized depolarization of the cells and inhibit repolarization at the reference electrode site. For example, the reference and recording electrodes 16 and 18 may be spaced apart a distance 42 of up to approximately 3 mm and each electrode 16 and 18 provided with a surface area less than approximately 15 mm². Obtaining a MAP signal reflective of local tissue electrophysiology requires both small electrode spacing and small electrodes. Small electrode spacing is required to localize the area of sensing and avoid averaging together regions with very different electrophysiological properties (e.g. conduction system with connective tissue). Small electrode size is required to ensure maximum differential between the two electrodes within this region.

In other embodiments, the reference and recording electrodes are spaced apart a distance 42 of up to approximately 1 mm, for example, less than approximately 0.5 mm. Recording electrode 18 may be provided with a length of approximately 0.1 mm and have a surface area of less than 0.2 mm² when provided as a ring electrode approximately 0.4 mm in diameter.

The proximal needle end 24 is electrically coupled to a monitoring device 32 used for recording, displaying and/or measuring MAP signals. The recording electrode is coupled to an insulated conductor wire 30 for delivering MAP signals to monitor 32. When hollow needle 14 is formed from an electrically conductive material, the proximal needle end 24 may be electrically coupled to monitor 32 for delivering the reference signal to monitor 32. In other embodiments, a separate insulated conductor wire may be provided for delivering the reference signal from reference electrode 16 to monitor 32. For example, hollow needle 14 may be formed of a non-conductive material, such as a polymeric material, with a reference electrode 16 attached at the distal tip 26 and recording electrode 18 attached proximally to distal tip 26 and spaced apart from the reference electrode 16. The proximal needle end 25 may be provided with an electrical connector assembly coupled to respective insulated conductors extending to reference electrode 16 and recording electrode 18 to facilitate electrical connection to electrodes 16 and 18.

The proximal needle end 24 may be fitted with a fluid coupling 34 to facilitate connection of needle 14 to a fluid reservoir 36. Fluid reservoir 36 may be embodied as a syringe, a fluid pump or other device capable of storing and delivering a fluid 38 to a targeted tissue site identified using MAP signals measured by needle 14. The fluid 38 stored by fluid reservoir 36 may be a drug solution, a viral vector solution for therapy, cell suspension, or other biological fluid delivered to provide a therapeutic effect at a targeted tissue site. In some embodiments, fluid 38 may be saline or alcohol delivered during a tissue ablation procedure as will be further described below. In general, fluid reservoir 36 is provided in fluid communication with the lumen of hollow needle 14 for delivering any fluid that may be needed during or as a part of a therapy delivery process.

The hollow needle 14 is used to both acquire MAP signals and deliver fluid to a tissue site. The system 10 thus allows localized MAP measurements to be made for precisely identifying a targeted therapy delivery site (e.g. a cardiac structure such as an SA or AV node, a localized area of diseased tissue, boundary of diseased or altered myocardial tissue, etc.) and delivering a fluid as a part of a therapy delivery process to the exact location in which the reference electrode is inserted. For example, a gene therapy may be delivered to slow AV nodal conduction to replace other drug therapies. A fluid is delivered to the insertion site of the needle 14 using needle 14, without having to withdraw needle 14, moving the catheter 12 or inserting a different therapy delivery device next to the catheter 12.

Monitor 32 may include a pulse generator 31 to deliver high voltage pulses to electrodes 16 and 18 to enhance uptake of the therapeutic fluids containing genetic material. Pulsed high voltage stimulation applied in conjunction with fluid delivery produces electroporation of local cells that creates transiently open pores in the cell membranes through which the genetic material is able to pass more freely. The effect of delivering such pulsed voltage at the delivery site is to deliver higher local concentrations of the genetic material into the targeted cells. Applied voltages of approximately 100-1000 Volts/cm are required for such an effect. Such an energy delivery in conjunction with delivery of the therapeutic agent can be considered to be an enhancement of the therapy.

The catheter 12 may be designed to function as a delivery catheter having a size, stiffness, maneuverability, steering mechanisms or other features that enable it to be advanced to an endocardial location. Catheter 12 may be advanced to an endocardial location with the use of a guide wire. Hollow needle 14 may inserted through an open lumen in catheter 12 before or after advancing catheter 12 to the endocardial location. If hollow needle 14 is present within a lumen of catheter 12 during advancement of the catheter 12 to an endocardial site, hollow needle 14 may remain retracted within catheter 12 during advancement and then extended outward from the distal opening 25 to make measurements in the endocardium.

In other embodiments, catheter 12 and needle 14 may be provided as a unitary device that is guided to an endocardial site using a separate guide catheter (not shown). Needle 14 may be fixedly positioned within catheter 12 having a fixed length of the needle 14 extending outward from catheter 12. The catheter 12 would then remain within a guide catheter until it reaches a desired measurement site.

FIG. 2 is an enlarged view of a distal end of a hollow needle 114 according to an alternative embodiment. Needle 114 is formed from an electrically conductive material and includes an electrically insulative coating or outer layer 120. Needle 114 is provided with a sharpened distal tip 126 and an exposed reference electrode 116 in an uninsulated portion of needle 114 extending proximally from distal tip 126. Recording electrode 118 is fixedly coupled to the outer layer 120 and electrically coupled to a conductor wire 130 extending to a proximal end of the needle 114. Conductor 130 may be an insulated conductor extending along an outer surface of outer layer 120 or be embedded in outer layer 120 or advanced through a lumen provided in outer layer 120.

In this embodiment, recording electrode 118 is provided as a point or button electrode attached to one side of the needle 114, instead of extending circumferentially around the needle in the form of a ring electrode. It is recognized that one or more recording electrodes 118 may be positioned at various circumferential positions around needle 114. When multiple electrodes are spaced around the circumference of needle 114, the multiple electrodes may be individually selectable, each having electrically isolated conductors, or they may be electrically coupled to a single electrical conductor to function as a single electrode. Multiple recording electrodes may alternatively or additionally be positioned at various longitudinal positions along needle 114. Multiple electrodes spaced from reference electrode 116 at different longitudinal distances and each coupled to a respective electrically insulated conductor allows different reference electrode-to-recording electrode spacings to be selected. Different MAP signal-to-noise ratio may be obtained by selecting different recording electrode-to-reference electrode combinations and an optimal electrode spacing identified. An optimal electrode spacing will provide the best signal-to-noise ratio and still record a localized MAP signal having a morphology similar to a single cell action potential.

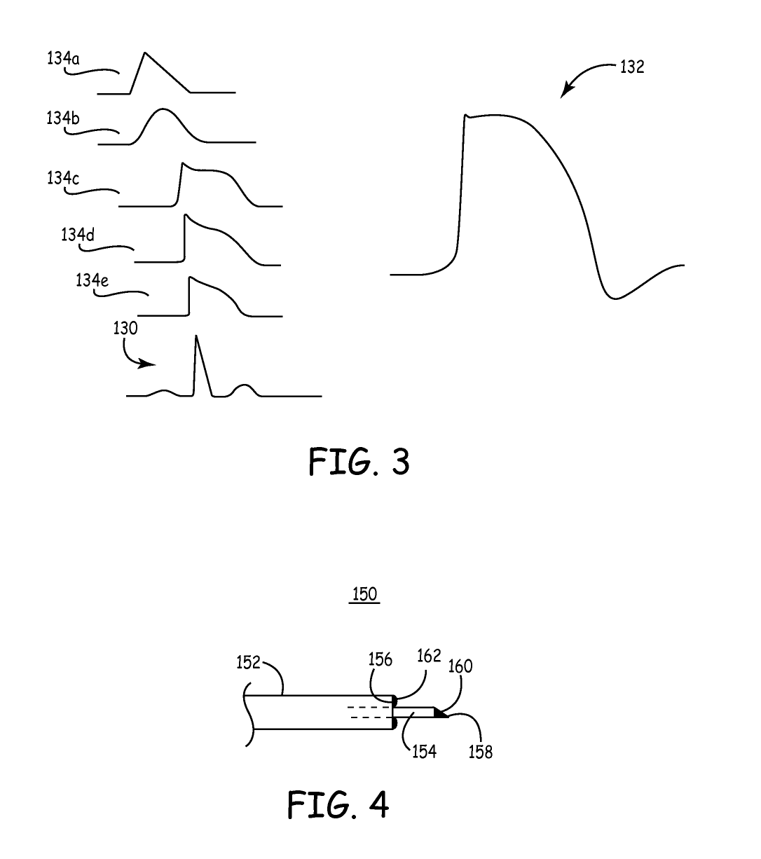

FIG. 3 is an illustration of an EGM signal 130 and a MAP signal 132. EGM signal 130 resembles an ECG signal and in general represents the spatial averaging of action potential signals from a large volume of cardiac tissue. Illustrative action potential signals from atrial muscle 134a, AV node 134b, Purkinje fiber 134c, upper ventricular myocardium 134d and lower ventricular myocardium 134e are shown. EGM signal 130 represents the spatial averaging of these various action potential signals arising from different parts of the heart and the contribution of these different action potential signals to the resulting EGM signal will depend on the location of the EGM sensing electrodes. While a bipolar EGM signal may be more localized than a unipolar EGM signal, the bipolar EGM signal still represents a spatially averaged signal over a larger surface area of the myocardial tissue and does not capture the electrical detail of an MAP signal.

In contrast, a MAP signal 132 recorded in the ventricle will have a morphology very similar to a ventricular action potential signal, e.g. 134d or 134e. The MAP signal 132 is a localized signal that averages a relatively much smaller volume of cardiac action potential signals and thus represents the local depolarization of the tissue instead of a spatial summation of depolarization over different regions of the heart.

FIG. 4 is an enlarged view of a distal end of a catheter 150 including MAP measurement electrodes according to an alternative embodiment. Catheter 150 includes an elongated tubular body 152 having a lateral distal face 156 and an internal lumen through which hollow needle 154 extends. Hollow needle 154 is provided with a sharpened distal tip 158 at which a reference electrode is formed as an uninuslated portion of hollow needle 154. A recording electrode 162 is positioned on the lateral distal face 156 of catheter body 152.

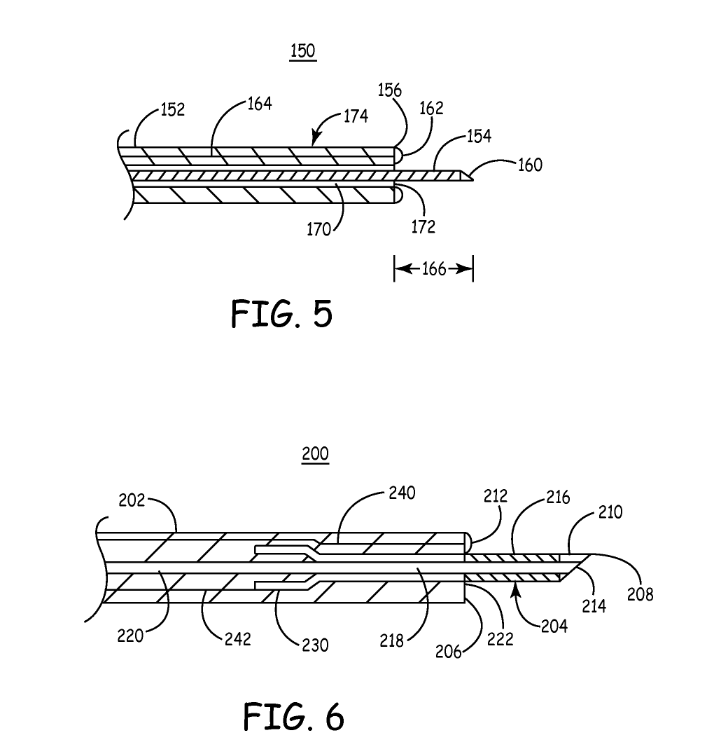

FIG. 5 is a sectional view of catheter 150. Lumen 170 extends through catheter body 152 from a proximal end (not shown) to an opening 172 in distal lateral face 156. Hollow needle 154 may be fixedly positioned within lumen 170 such that a distance 166 between reference electrode 160 and recording electrodes 162 is fixed. Alternatively, needle 154 may be advanceable and retractable within catheter body 152 such that the distance 166 is a variable distance controlled by a user advancing or retracting hollow needle 154 at a proximal needle end. The proximal needle end may include markings, grooves or other features for controlling the advancement or retraction of needle 154 a known distance out from lateral distal face 156 such that the reference electrode-to-recording electrode distance 166 is known.

Recording electrode 162 is shown as a ring electrode that extends concentrically around needle 154 and is electrically insulated from needle 154, e.g. by an insulative outer coating applied to needle 154. In alternative embodiments, recording electrode 162 may be provided as a small button or point electrode positioned at any radial location along distal lateral face 156. In still other embodiments, recording electrode 162 may be provided as a ring, button or point electrode positioned at a circumferential location along the outer longitudinal surface 174 of catheter body 152, proximate the catheter distal end face 156 such that the interelectrode distance 166 does not become too large to acquire MAP signals.

An electrical conductor 164 is electrically coupled to recording electrode 162 and extends through catheter body 152 to the proximal catheter end. As described previously, any of the catheter systems described herein may include multiple recording electrodes each electrically coupled to a respective conductor to enable selection of different recording electrode sizes and/or distances from reference electrode 160.

FIG. 6 is a sectional view of a distal end of a catheter 200 according to an alternative embodiment. Catheter 200 includes an elongated tubular body 202 defining an inner lumen 220 extending from a proximal catheter end to an opening 222 in the distal lateral face 206 of catheter body 202. A recording electrode 212 is shown positioned on distal lateral face 206. Recording electrode 212 is electrically coupled to an insulated conductor 240 extending through catheter body 202 to a proximal catheter end.

Hollow needle 204 includes a sharpened distal tip 208 at which an uninsulated portion of needle 204 forms a reference electrode 210. The remaining portion of needle 204 extending out from opening 222 is electrically insulated by an outer layer or coating 216. Hollow needle 204 is electrically coupled to a conductor 242 that extends within catheter body 202 to the catheter proximal end to facilitate electrical connection to reference electrode 210.

Hollow needle 204 defines an inner lumen 218 in fluid communication with catheter lumen 220 and extending to a distal opening 214 of needle 204. In this embodiment, hollow needle 204 does not extend along the entire length of catheter body 202 as described above in reference to other embodiments. Hollow needle 204 extends partially along the length of catheter body 202 and may be adhesively bonded or press fit within inner lumen 220 of catheter 200. Alternatively, hollow needle 204 is overmolded during a manufacturing process performed to form catheter body 202. Hollow needle 204 may include a widened portion forming a flange 230 that becomes solidly embedded in the polymeric material used to form catheter body 202 to securely fix hollow needle 204 within catheter 200.

In this embodiment, the proximal end of catheter 200 is coupled to a fluid reservoir 36 (FIG. 1) to enable fluid to be delivered through catheter lumen 220, needle lumen 218 and out needle opening 214. The proximal catheter end is also provided with appropriate electrical connectors for enabling electrical connection to electrodes 210 and 212 via conductors 240 and 242 for recording MAP signals. MAP signals are used to identify a targeted tissue site for delivering a therapy that includes fluid delivery to the tissue site via lumens 220 and 218.

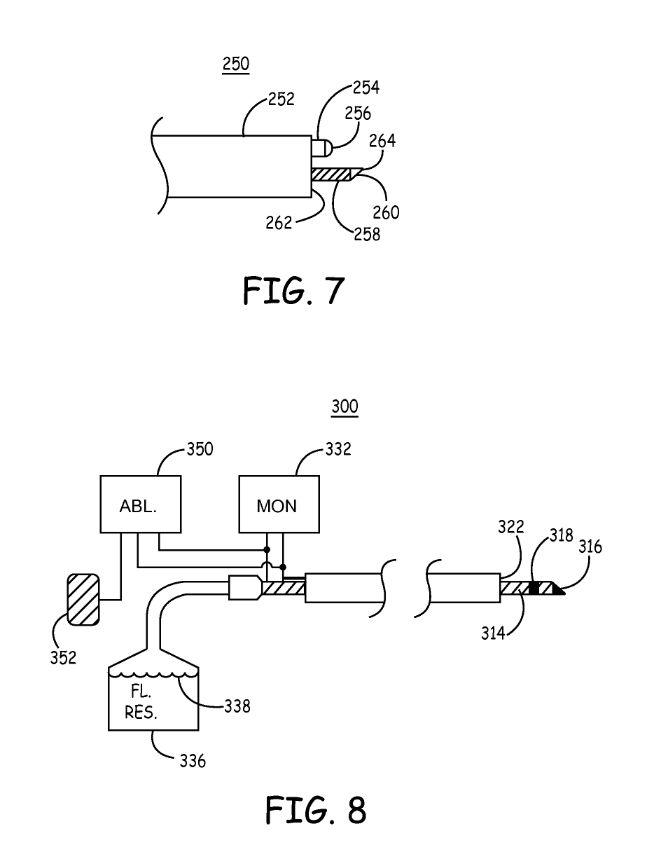

FIG. 7 is an enlarged plan view of a distal end of a catheter 250 including an elongated catheter body 252. A hollow needle 258 extends outward from a distal lateral face 262 of catheter body 252. The hollow needle functions as a reference electrode 260 having a distal sharpened end 264 for causing a controlled amount of tissue injury to depolarize and inhibit repolarization of adjacent cells. A recording probe 254 extends from distal lateral face 262 and includes a recording electrode 256. One or both of recording probe 254 and hollow needle 258 may be advanceable and retractable in catheter body 252. Alternatively, both probe 254 and needle 258 may extend fixed distances from catheter body 252.

FIG. 8 is a schematic view of a catheter system for measuring MAP signals and delivering an ablation therapy. In system 300, a monitor 332 is electrically coupled to reference electrode 316 and recording electrode 318 provided on a hollow needle 314 for measuring MAP signals. MAP signals are used to identify a targeted location for delivering an ablation therapy, such as the AV node, pulmonary vein, atrial myocardium, ventricular myocardium, or other locations.

After identifying a tissue site, an ablation energy source 350 is used to deliver ablative energy to the tissue site. The ablation energy source 350 may be coupled to the reference and recording electrodes 316 and 318 respectively to deliver bipolar ablation energy between the two electrodes 316 and 318 to form a highly localized lesion. A fluid 338 stored in fluid reservoir 336 may be delivered during or in association with the ablation procedure, e.g. delivering a saline to provide tissue cooling. Chemical ablation could alternatively be performed using alcohol delivered from fluid reservoir 336.

Alternatively, ablation energy may be delivered in a unipolar manner between a surface patch electrode 352 and either of the reference electrode 316 or the recording electrode 318. In another ablation delivery configuration, the reference electrode 316 and recording electrode 318 are electrically coupled together at the ablation energy source 350 to form a single ablation electrode delivering unipolar ablation energy between the combined electrodes 316 and 318 and a patch electrode 352. In this way, ablation therapy can be performed precisely at a tissue site identified using MAP and a highly localized lesion can be created.

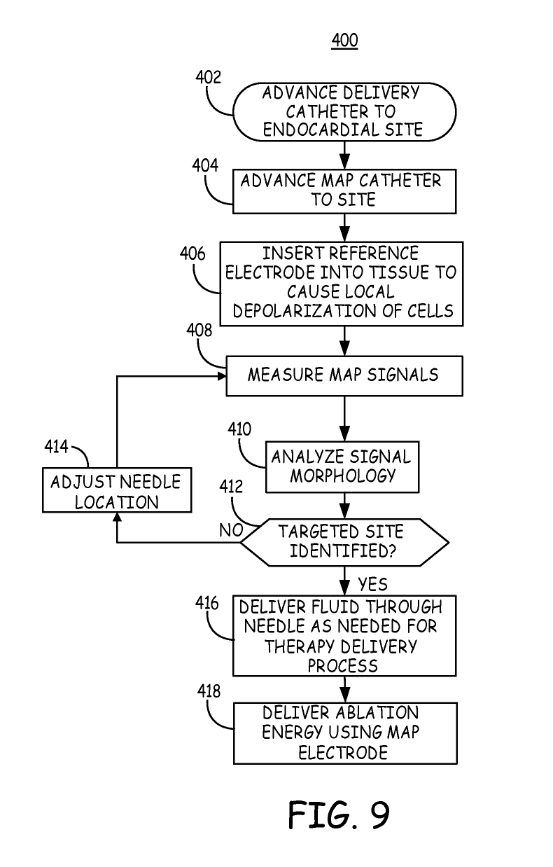

FIG. 9 is a flow chart of a method for delivering a therapy. Flow chart 400 is intended to illustrate the functional operation of the device, and should not be construed as reflective of a specific form of software or hardware necessary to practice the methods described. Portions of the method described in conjunction with flow chart 400 may be implemented in a computer-readable medium that includes instructions for causing a programmable processor to carry out the methods described. A “computer-readable medium” includes but is not limited to any volatile or non-volatile media, such as a RAM, ROM, CD-ROM, NVRAM, EEPROM, flash memory, and the like. The instructions may be implemented as one or more software modules, which may be executed by themselves or in combination with other software.

At block 402 a delivery catheter is advanced to a desired endocardial site, which may be within an atrial or ventricular chamber. At block 404, the MAP measurement catheter is advanced to the endocardial site through the delivery catheter. The MAP measurement catheter is advanced out from the delivery catheter to enable insertion of the hollow needle tip into tissue at the endocardial site. The MAP reference electrode located at the needle tip is thereby positioned at a site of locally depolarized tissue due to tissue injury of limited size caused by insertion of the needle tip.

At block 408, MAP signals are measured using the recording electrode and the reference electrode of the MAP measurement catheter. The MAP signals are analyzed at block 410 to determine if the MAP signal morphology is indicative of a targeted therapy delivery site. The MAP signal morphology that corresponds to a targeted therapy delivery site will depend on the particular therapy delivery application.

In one embodiment, the MAP signal is analyzed to determine if the morphology corresponds to a particular cardiac structure, e.g. the AV node. If the morphology is not indicative of the AV node, the hollow needle is withdrawn, repositioned (block 414) and new MAP signals are obtained at a new location until a location is identified that results in the expected MAP signal morphology.

In other embodiments, a mapping procedure may be performed in which the MAP signals are measured at block 408, analyzed at block 410 and the needle location is then adjusted at block 414 to obtain MAP signals at a new location. This process is repeated until an area of the myocardium has been mapped. The MAP signal duration or other signal features (e.g. upstroke velocity, repolarization) may be measured for the different measurement sites to identify areas having significantly different MAP signal morphology. A significantly different MAP signal morphology may be evidence of a pathological or biological process occurring within an identified boundary, such as inflammation, fibrosis, formation of autonomic ganglia which may become a substrate for arrhythmias or other processes that may lead to a pathological cardiac condition or arrhythmias.

By precisely identifying a structure or area, an appropriate therapy can be selected and delivered at block 416. A gene therapy may be delivered using the hollow needle of the MAP measurement catheter directly to the location of the MAP signal measurements. Other biological or pharmacological fluids may be delivered as a part of a therapy delivery process. As described previously, pulsed high voltage electrical stimulation may be delivered via the MAP electrodes in conjunction with the fluid delivery to produce electroporation. In other embodiments, the selected therapy may include delivery of ablative energy using one or both of the MAP reference and recording electrodes at block 418.

Thus, an apparatus and associated methods are described for identifying a targeted therapy delivery site using MAP signal measurements and for delivering a therapy to the identified site using the MAP measurement catheter. The apparatus and methods have been presented in the foregoing description with reference to specific embodiments. It is appreciated that various modifications to the referenced embodiments may be made without departing from the scope of the disclosure as set forth in the following claims.

1. A system for measuring monophasic action potential signals for identifying a targeted tissue location and delivering a therapy to the targeted tissue location, the system comprising:
a hollow needle having a sharpened distal tip and comprising a first electrode at the distal tip and a fluid delivery lumen extending through the needle from a proximal needle end to an opening in the sharpened distal tip, the sharpened distal tip sized to cause tissue injury at a reference tissue site for depolarizing the tissue at the reference tissue site and inhibit repolarization;
a second electrode spaced apart from the first electrode and electrically insulated from the first electrode for measuring of a monophasic action potential;
a monitor electrically coupled to the first electrode and the second electrode for recording monophasic action potential signals for identifying the target tissue location for delivering the therapy; and
a fluid reservoir in fluid communication with the fluid delivery lumen for delivering a fluid contained in the reservoir to the targeted tissue location during therapy delivery.

2. The system of claim 1 wherein the second electrode is positioned along the hollow needle proximally from the first electrode.

3. The system of claim 2 wherein the second electrode extends circumferentially around an outer diameter of the hollow needle.

4. The system of claim 1 further comprising:
an outer insulative layer, the hollow needle extending through the outer insulative layer;
wherein the second electrode is positioned on the outer insulative layer.

5. The system of claim 1 further comprising:
an outer insulative layer, the hollow needle extending through the outer insulative layer; and
an elongated probe having a blunted distal end and a proximal end, the second electrode located at the probe distal end, the elongated probe extending through the outer insulative layer.

6. The system of claim 1 further comprising an elongated delivery catheter having an open lumen extending from a proximal catheter end to a distal catheter end, the open lumen in fluid communication with the lumen of the hollow needle, the hollow needle extending along only a distal portion of the delivery catheter lumen.

7. The system of claim 1 further comprising an ablation energy source coupled to one of the first electrode and the second electrode for delivering ablative energy to the targeted tissue location.

8. The system of claim 5 further comprising a surface patch electrode coupled to the ablation energy source for delivering unipolar ablative energy to the targeted tissue location.

9. The system of claim 1 wherein the first electrode and the second electrode each have a surface area of up to approximately 15 square millimeters.

10. The system of claim 1 wherein the first electrode and the second electrode are spaced up to approximately 1 mm apart.

11. The system of claim 1 wherein the distal tip having an outer diameter less than approximately 1 mm.

12. The system of claim 1 further comprising a plurality of electrodes spaced apart from the first electrode, each electrically coupled to a respective insulated conductor,
wherein the monitor is electrically coupled to the first electrode and selectively coupled to one of the plurality of electrodes spaced apart from the first electrode for recording monophasic action potential signals at an optimal interelectrode distance.

13. The system of claim 1 further comprising a processor for comparing a monophasic action potential signal to an expected signal for identifying the target tissue location.

14. The system of claim 1 further comprising an electrical pulse generator coupled to the first and second electrodes for delivering high voltage electrical pulses during the fluid delivery.

15. A method for measuring monophasic action potential signals for identifying a targeted tissue location and delivering a therapy to the targeted tissue location, the method comprising:
advancing a hollow needle to a cardiac location, the hollow needle having a sharpened distal tip and comprising a first electrode at the distal tip and a fluid delivery lumen extending through the needle from a proximal needle end to an opening in the sharpened distal tip;
inserting the sharpened distal tip into tissue at the cardiac location to cause tissue injury at a reference tissue site for depolarizing the tissue at the reference tissue site and inhibiting repolarization;
electrically coupling the first electrode and a second electrode spaced apart from the first electrode and electrically insulated from the first electrode to a monitor;
measuring of a monophasic action potential from the first electrode and the second electrode;
identifying the target tissue location for delivering the therapy in response to the measured monophasic action potential; and
delivering a fluid to the targeted tissue location via the hollow needle.

16. The method of claim 15 further comprising advancing a delivery catheter to the cardiac location and advancing the hollow needle out from a distal end of the delivery catheter.

17. The method of claim 15 further comprising advancing an elongated probe having a blunted distal end to the cardiac location, the second electrode located at the probe distal end.

18. The method of claim 15 further comprising coupling an ablation energy source to one of the first electrode and the second electrode and delivering ablative energy to the targeted tissue location.

19. The method of claim 18 further comprising coupling a surface patch electrode to the ablation energy source and delivering unipolar ablative energy to the targeted tissue location.

20. The method of claim 15 further comprising selectively coupling the monitor to each of a plurality of electrodes spaced apart from the first electrode for recording monophasic action potential signals at different interelectrode distances and selecting one of the plurality of electrodes for recording MAP signals at an optimal interelectrode distance.

21. The method of claim 15 further delivering high voltage electrical pulses via the first and second electrodes during the fluid delivery.
